(19) United States
(12) Patent Application Publication
(10) Pub. No.: US 2002/0182594 A1
(43) Pub. Date: Dec. 5, 2002

(54) Chemomagnetic retrieval of CMV and CMV infected cells

(75) Inventors: Thomas J. Schall, Menlo Park, CA (US); Mark E.T. Penfold, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ChemoCentryx, San Carlos, CA (US)

(21) Appl. No.: 10/061,944

(22) Filed: Feb. 1, 2002

Related U.S. Application Data
(60) Provisional application No. 60/266,094, filed on Feb. 2, 2001.

(51) Int. Cl. C12Q 1/70; G01N 33/53

(52) U.S. Cl. 435/5; 435/7.1

(57) ABSTRACT

Methods and apparatus are provided herein for collecting CMV and/or CMV infected cells from a host infected with CMV. Such methods and apparatus have utility in tracking the dissemination or infection of the host, use as an in vivo or ex vivo collection mechanism to measure mutation rates and selective pressures after in vivo passage, and in therapeutic treatments in which CMV and/or CMV infected cells are removed from a host.

### CROSS-REFERENCES TO RELATED APPLICATIONS

This application claims the benefit of U.S. Provisional Application No. 60/266,094, filed Feb. 2, 2001, which is incorporated herein in its entirety for all purposes.

### STATEMENT AS TO THE RIGHTS TO INVENTIONS MADE UNDER FEDERALLY SPONSORED RESEARCH AND DEVELOPMENT

This invention was partially made with government support under Grant Number N66001-01-C-8009 awarded by the Defense Advanced Research Projects Agency (DARPA) of the Department of Defense. The government may have certain rights in this invention.

### BACKGROUND

Cytomegaloviruses (CMVs) are common pathogens and are members of the , subgroup of the herpesvirus family. CMV is a slow replicating, species-specific complex DNA virus found in most mammals. CMV has adopted subtle evolutionary strategies for evading the immune system of an infected host, while disseminating through the host tissues.

The genome (230 kb) of human CMV (HCMV) includes a long and short unique region (UL and US, respectively), each of which is flanked by inverted repetitions. The entire HCMV genome has been sequenced (Chee, M. S., et al. (1990) Curr. Top. Microbiol. Immunol. 154:125-169) and appears to contain over 200 open reading frames.

One of these open reading frames is referred to as US28, which encodes a protein (also “US28”) that acts as a functional receptor for certain human and viral chemokines (see, e.g., Gao & Murphy, 1994, J Biol. Chem. 269:28539-42). Upon infection of a cell by CMV, US28 is expressed on the surface of the infected cell and becomes capable of responding to chemokines in the environment. Three other open reading frames called US27, UL33 and UL78 encode for proteins having homology to US28 as shown in Table 1 below.

TABLE 1
Exemplary Viral Chemokine Elements and
Immune-inhibitory Genes

| CMV Chemokine | | |
|---|---|---|
| Elements or | GenBank | |
| Immune- | Accession | |
| inhibitory Genes | No. | Reference |
| US27 | X17403 | Chee et al, 1990, Nature, 344:774 |
| US28 | L20501, | Neote et al, 1993, Cell, 72:415-25 |
| | AF073831-35 | |
| UL33 | X53293 | Chee et al, 1990, Nature, 344:774 |
| UL78 | X17403 | Chee et al, 1990, Nature, 344:774 |

Chemokine receptors such as US28 generally are G protein coupled receptors. Structurally these receptors have seven transmembrane segments that loop in and out of the cell membrane, as well as an intracellular tail that is coupled to a G protein signal transducing molecular complex.

The chemokines themselves constitute a subgroup of a larger class of signaling proteins and have the ability, among other things, to promote cellular migration (Zlotnik et al. (1999) Crit. Rev. Immunol. 19:1-47). The chemokines generally are divided into four groups based upon the arrangement of certain cysteine residues within the protein that can form disulfide bonds. One class of chemokines is the beta chemokines that are characterized by having two adjacent cysteines; this structure is referred to in shorthand form simply as CC. The beta chemokines are involved in attraction of monocytes and leukocytes. The alpha chemokines, in contrast, have a single amino acid separating the two cysteine residues, and thus their structure is designated as CXC. These chemokines are primarily involved in attracting polymorphonuclear cells. The fractalkines or delta-chemokines constitute a third class of chemokines and tend to be cell bound molecules. The two cysteines in this class are separated by three amino acid residues, a structure designated as CX3C. This class of chemokines are expressed at high levels in the brain; some evidence indicates that the fractalkines are involved in neuron-glial cell interactions (see, e.g., Harrison, et al. (1998) Proc. Natl. Acad. Sci. U.S.A. 95:10896-10901; and Nishiyori, A. et al. (1998) FEBS Lett. 429:167-172). The US28 receptor of HCMV is characterized in part by its very strong affinity for fractalkine. The structure of the final class of chemokines is simply referred to as C (also gamma-chemokines), because these chemokines contain only a single N-terminal cysteine involved in a disulfide bond. The chemokine receptors have varying specificity for the different classes of chemokines. Some chemokine receptors can bind chemokines from different classes.

### SUMMARY

A variety of apparatus and methods are provided herein for collecting CMV and/or CMV infected cells from a patient infected with CMV. Such apparatus and methods have utility in tracking the dissemination or infection of the host, use as an in vivo or ex vivo collection mechanism to measure mutation rates and selective pressures after in vivo passage, and in therapeutic treatments in which CMV and/or CMV infected cells are removed from the diseased patient. In some instances, the compound utilized in the apparatus and methods is a ligand for US28. Such compounds are useful because the evidence indicates that this molecule is expressed on the surface of both virions and infected cells and is involved in viral dissemination by binding to various chemokines.

Certain apparatus that are provided comprise a collector that includes a compound that binds CMV or the CMV infected cell and a circuit (i) adapted for connection to the blood system of a patient, (ii) adapted for the flow of withdrawn blood therethrough, and (iii) in fluid communication with the collector. In some apparatus, the circuit comprises an outlet line and a return line, each in fluid communication with the blood system of the patient. The circuit then is adapted for withdrawal of blood from the patient's blood system via the outlet line, passage of the blood through the collector and the return of the blood to patient's the blood system via the return line. Typically, the compound is attached (e.g., via an optional linker) to a support material; the resulting complex is then placed in the collector such that passage of blood is facilitated.

Other collection devices for collecting CMV and/or CMV infected cells comprise a support and the compound that binds CMV and/or the CMV infected cell. Certain such devices are implant devices that are adapted for insertion into a diseased patient. Such implants can be composed of a variety of materials but in some instances are made of an absorptive material. A specific example of such materials is a surgical sponge. Other devices, however, are patches that are adapted for placement on the skin of the infected patient. In this form, the compound is absorbed or impregnated into the patch.

The collection devices and apparatus that are provided can be utilized in a variety of applications. Certain methods for collecting cytomegalovirus (CMV) or CMV infected cells involve contacting an infected patient's blood or tissue that contains CMV or a CMV infected cell with a compound that binds CMV, whereby CMV or the CMV infected cell in the blood or the tissue is collected or bound by the compound. In related methods, the contacting process involves withdrawing blood containing CMV or the CMV infected cell from the patient and flowing the blood through or into a collector that contains the compound, whereby CMV and/or the CMV infected cell binds to the compound in the collector. Optionally, the withdrawn blood is recirculated back into the patient. Methods in which the blood is recirculated can be conducted such that the withdrawing, contacting and recirculating steps are performed continuously or non-continuously.

Collection methods utilizing an implantable support involve inserting a support comprising a compound that binds CMV and/or the CMV infected cell into the blood system of a patient infected with CMV such that blood in the blood system contacts the compound, whereby CMV or a CMV infected cell in the blood is collected at the support. Typically, the inserted support is subsequently removed from the patient after CMV and/or CMV infected cells have accumulated at the implant device.

By virtue of the fact that the collection method collect CMV and CMV infected cells, the methods can be utilized therapeutically to treat animals suffering from CMV infection. Such treatment methods are not limited to treatment of humans, but can also be utilized, for example, with various animals that serve as models for methods with humans (e.g., non-human primates). However, the collection methods can be utilized for a number of non-therapeutic purposes as well.

For example, the collection methods can also be used to monitor the presence and rate of mutations in CMV; such information can be useful in detecting formation of resistance to various pharmaceutical agents, for instance. Certain of the provided methods involve collecting CMV and/or at least one CMV infected cell from a patient infected with CMV by contacting the blood or a tissue of the patient with a compound that binds CMV and/or a CMV infected cell, whereby CMV or at least one CMV infected cell is bound from the blood or tissue. One then detects the presence and/or absence of a mutation in CMV obtained from the collected CMV or CMV infected cell. In certain methods designed to assess development of CMV resistance, if a mutation is detected, then it is determined whether the mutation confers resistance to a pharmaceutical agent.

As noted supra, certain of the methods and apparatus provided herein are able to collect or retrieve CMV or CMV infected cells by utilizing compounds that bind to US28. A number of small organic compounds having such activity have been identified by the present inventors and are described in detail infra.

### BRIEF DESCRIPTION OF THE FIGURES

FIG. 1 is a schematic representation of one example of an external collection device for collecting CMV or CMV infected cells from an infected patient.

### DESCRIPTION

I. Definitions

As used in this specification and the appended claims, the singular forms “a,” “an” and “the” include plural references unless the content clearly dictates otherwise.

Unless defined otherwise, all technical and scientific terms used herein have i: i;the meaning commonly understood by a person skilled in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF AS SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

Abbreviations: CMV, cytomegalovirus; S(−)-IBZM, S(−)-34odo-2-hydroxy-6-methoxy-N[(1-ethyl-2-pyrrolidinyl)methyl]-benzamide.

The following definitions are provided to assist the reader in the practice of the invention.

As used herein, the term “cytomegalovirus (CMV)” has the normal meaning in the art and refers to one of a family of double stranded DNA viruses of the betaherpes group with positional and genomic similarity to human herpes virus 5 (cytomegalovirus) including, without limitation, human CMV AD169 (ATCC # VR 538), human CMV Towne (ATCC # VR 977), human CMV Davis (ATCC # VR 807), human CMV Toledo (Quinnan et al, 1984, Ann Intern Med 101: 478-83), monkey CMV Rh68.1 (ATCC # VR 677), monkey CMV CSG (ATCC # VR 706), rat CMV Priscott (ATCC # VR 991), mouse CMV Smith (ATCC # VR 1399) and others. “ATCC” is the American Type Culture Collection, 10801 University Boulevard, Manassas, Va. 20110-2209, USA. The 230-kb dsDNA genome of human and murine CMV were sequenced (see, e.g., Chee et al., 1990, Curr. Top. Microbiol. Immunol. 154:125-169; also see Rawlinson, 1996, J Virol. 70:8833-49, both incorporated herein in their entirety).

Various open reading frames from human CMV (HCMV) are referred to herein using the nomenclature of Chee et al [e.g., US28, US33, US78 (human US28, human US33, human US78, respectively)]. In general, reference to such reading frames from HCMV also refer to the sequences of sequence and positional homologs of such reading frames found in different HCMV strains, including sequences in any naturally occurring HCMV strain, and mutations to such strains. In some instances the term can also refer to various splice variants not yet characterized in the literature.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(l,4-pentadienyl), ethynyl, 1- and 3-propynyl, 3-butynyl, and the higher homologs and isomers. When used alone, the term “alkyl” refers to unsubstituted versions of the radicals indicated above. Substituted forms of “alkyl” are defined in more detail below.

The term “alkylene” by itself or as part of another substituent means a divalent radical derived from an alkane, as exemplified by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(0)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl or heterocyclyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “(C₁-C₄)haloalkyl” is mean to include trifluoromethyl, 2,2,2-trifluoroethyl, 4-chlorobutyl, 3-bromopropyl, and the like.

The term “acyl” is used in its conventional sense and refers to an organic radical derived from an organic acid by the removal of the hydroxyl group. Examples of “acyl” groups include acetyl, propionyl, butanoyl, hexanoyl, isobutyryl, octanoyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quatemized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, l-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH— C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R″′ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, ,—NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula —T—C(O)—(CH₂)_{q}—U—, wherein T and U are independently —NH—, —O—, —CH₂—or a single bond, and q is an integer of from 0 to 2. Alternatively, two of the substituents on adjacent atoms of the aryl or heteroaryl ring may optionally be replaced with a substituent of the formula —A—(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fuimaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds disclosed herein can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds which are provided possess asymmetric carbon atoms (optical centers) or double bonds; the racemates, diastereomers, geometric isomers and individual isomers are all intended to be encompassed within the scope of the present invention.

The compounds described herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

A “patient” or “host” refers broadly to an animal that is infected with CMV. -0 p The term includes animals of a variety of types including mammals and non-mammals, such as humans, non-human primates (e.g., monkeys, chimpanzees, apes and gorillas) and commercial livestock (e.g., chickens, bovines, sheep, porcines and the like).

The term “tissue” has its usual meaning in the art and refers to a collection of similar cells typically having a particular function.

A “pharmaceutical agent,” “drug” or “pharmaceutically active agent” are used interchangeably and refer to a chemical substance suitable for delivery to an animal that induces a desired effect. Such substances are often used in the prevention, diagnosis, alleviation, treatment and/or cure of a disease. The term includes agents that are therapeutically effective as well as agents that are prophylactically effective.

The term “contact” or “contacting” when used in reference to contact between CMV or CMV infected cells with a compound is meant broadly to refer to direct contact, as well as indirect contact, between CMV or CMV infected cells with the compound. An example of indirect contact, for example, is contact that occurs when blood containing virus or a virus infected cell diffuses throught a tissue (e.g., skin) to come in contact with the compound).

The term “mimetic” has its usual meaning in the art and refers to a compound whose structure and/or chemical characteristics accord it an activity similar to another (e.g., reference) compound.

II. Overview

Apparatus and methods for retrieving or concentrating CMV or CMV infected cells from a host infected with CMV are provided herein. In general such apparatus and methods utilize a compound that is able to bind selectively a molecule displayed on the exterior surface of CMV, or a molecule displayed on the surface of a CMV infected cell that correlates with CMV infection. Thus, methods and devices for concentrating CMV or CMV infected cells in vivo and/or removing same from a host infected with CMV.

As indicated in the Background section, the CMV genome contains an open reading frame designated US28 that encodes a protein that acts as a functional receptor for certain human and viral chemokines. Upon infection of a cell by CMV, US28 is expressed on the surface of the infected cell and becomes capable of responding to chemokines in the environment. Certain of the inventors have also shown that US28 is expressed on virions (see, e.g., PCT Application No. 01/23792, filed on Aug. 30, 2001, entitled “Inhibition of CMV Infection and Dissemination,” and having attorney docket number 019934-002510PC). Since the CX3C chemokine, fractalkine, is expressed on certain endothelial cell surfaces and on populations of dendritic cells (DC) and binds with very high affinity to US28 (K₁=approximately 50 pM), the evidence indicates that it defines a portal through which CMV infected cells or virions go from the circulation to the tissue space, as well as finding residence in dendritic cells. Certain of the apparatus and methods which are provided take advantage of this aspect of CMV infection to collect CMV or CMV infected cells. In particular, the apparatus and methods use US28 chemomimetics (i.e., compounds that mimic US28 ligand activity) to retrieve CMV or CMV infected cells from an infected host. Such compounds can be utilized to induce migration of US28-bearing cells or virions in vivo; alternatively, the compounds capture such cells or virions as they come in contact with the compound. The compounds utilized can be of any of a number of types, such as proteins, peptides, peptide mimetics and the like. As described in greater detail below, a number of specific US28 small organic molecule mimetics have been identified that can be utilized in the methods and apparatus that are disclosed herein.

Some devices that are provided are implant devices that include a support that contains a compound that binds CMV or a CMV infected cell (e.g., compounds that are ligands of US28). These implant devices are generally adapted for insertion into the CMV infected host, particularly for insertion such that the implant device is in contact with the blood of the infected host. Thus, CMV or CMV infected cells are induced to migrate to the compound-containing implant device and/or are captured as they flow past the implant device.

Other apparatus are external devices. Certain of these devices include a circuit for withdrawing blood from a host infected with CMV which is connected to a collector that contains the compound that binds CMV or a CMV infected cell. Typically, the circuit includes an outlet line and a return line, each attached to the collector. The outlet line is for withdrawing blood from the host and flowing the blood to the collector; the return line returns blood purified (i.e., blood with a reduced concentration of CMV or CMV infected cells) at the collector to the host.

As described further infra, the apparatus and devices disclosed herein can also be utilized for performing a variety of treatment methods and analyses. For instance, the apparatus can be utilized to track dissemination or infection of a host, used as an in vivo or ex vivo retrieval mechanism to measure mutation rates and selective pressures after in vivo passage, and to remove virus or recombinant virus from a host (e.g., for therapeutic purposes).

Because CMV strains infect essentially all mammals, the apparatus and methods that are disclosed herein can be utilized to collect CMV and CMV infected cells from a variety of animals, including, for example, humans, non-human primates and a variety of commercial livestock. Further, the CMV to be removed can be wild-type CMV, or genetically engineered CMV. In some instances, the CMV is a genetically engineered virus useful for stimulating in immune response in a host, e.g., as described in PCT Application No. ______, filed Feb. 1, 2002, entitled “Methods And Compositions Useful For Stimulating An Immune Response,” and having Attorney docket no. 019934-001610, which is incorporated herein by reference for all purposes.

III. Description of Collection Apparatus and Devices

A. Implant Devices

1. Design

The implant devices that are provided generally include a compound that binds CMV or a CMV infected cell and a support or substrate containing the compound. The word “containing” as used in this context is used broadly to indicate that the compound is associated with the support. Thus, by way of illustration but not limitation, the compound can be attached to a surface of the support, the compound can be absorbed by the support, or the compound can be linked to the support via a linker.

As indicated supra, the compound contained by the support is one which can bind selectively a molecule displayed on the exterior surface of CMV, or a molecule displayed on the surface of a CMV infected cell that correlates with CMV infection. In certain devices, the compound is a ligand for US28. A more specific discussion of compounds that are suitable for use with the implant device are described infra in section IV.

The support is adapted for insertion into a host or patient that is infected with CMV. Typically, the support is adapted for placement such that it is in contact with the blood system of the infected host. The term “contact” or “contacting” when referring to contact between the implant and the support is used broadly to refer to direct and indirect contact (e.g., diffusion of blood and/or virus through tissue to contact the support). The support can have various forms, including, by way of illustration but not limitation, sponges, microcapsules, beads and patches. The support can be made of variety of different materials, so long as the final material is sterile and compatible with the bodily fluids (e.g., blood) in which it will be in contact. In some instances, the support is quite rigid; this may be the case, for example, if the compound is attached or linked to the support. Suitable supports in such instances can be formed of various plastics (e.g., cross-linked polystyrene, polyester and polyurethane) and latex, for example. Alternatively, if the compound is to be absorbed into the support, then the support is made of an absorptive material. As noted above, certain such supports are sponges. Examples of absorptive materials that can be utilized include, without limitation, gelfoam sponges (e.g., those available from Henry Schein, Port Washington, New York and from Pharmacia Upjohn), polyester sponges, polyurethane foam, Matrigel (Beckton Dickinson) and absorptive materials composed primarily of extracellular matrix proteins and the like. The size and shape of the support varies depending upon the size of the host and the location in which the support is to be placed.

2. Mode of Operation

In general, the implant device that contains the compound is inserted into a host or patient infected with CMV such that the support is in contact with the blood system of the patient. When positioned in this manner, the implant device can capture CMV or CMV infected cells from the blood. Without intending to be limited to a particular mechanism, capture can occur by promoting the migration of CMV or CMV infected cells to the implant device and/or as CMV and CMV infected cells are brought in contact with the implant device as they pass by the implant device. The support may also be placed in contact with an infected tissue.

Prior to insertion, the implant device can be prepared in various ways. For instance, the compound may be chemically coupled to the support using chemistries and optionally linkers that are well known in the art. Absorptive supports are first contacted with the compound to allow for absorption of the compound. In such instances, the compound is typically mixed with various pharmaceutically acceptable carriers, diluents or excipients to facilitate absorption of the compound into the support (e.g., sponge). These are selected not to interfere with the binding of the compound to CMV or a CMV infected cell. Examples of suitable agents that can be used to mix with the compound include, but are not limited to, distilled water, physiological saline, PBS, Ringer's solution, dextrose solution, and Hank's solution. Additional agents such as pH adjusting and buffering agents, toxicity adjusting agents and wetting agents may also be utilized in the mixture.

The insertion process itself is conducted under aseptic conditions. The host is typically at least partially, if not completely, anesthetized prior to insertion of the implant device. The support can be placed in a number of locations, but is generally positioned to be in contact with the blood system of the host. Thus, the implant device can be positioned subcutaneously or intraperitoneally, for example.

The implant device is left within the infected host for a sufficient time period to enable virions and/or CMV infected cells to migrate to the support or flow past the support such that virus and/or cells are collected at the implanted device. The support can then be removed for analysis or simply to remove the collected virions and/or infected cells from the host, thereby lessening the level of infection in the patient.

Methods providing specific guidance for insertion of a compound-containing sponge into an infected host are discussed by, for example: Ford-Hutchinson et al., 1984, Prostaglandins 28:13-27; Garrett et al., 1983, Ann Rheum Dis. 42:439-42; Iuvone et al., 1997, Br J Pharmacol., 121:1637-44; Middleton et al., 1989, J Leukoc Biol. 46:461-6; Mellor et al., 1986, 18:550-4; Fine et al., 2000, Inflammation 24:331-46).

B. External Apparatus

1. Design

In addition to the foregoing implant devices, various external apparatus are provided for collecting CMV and/or CMV infected cells from an infected host. In general, the design for such apparatus typically includes a collector that contains the compound that binds CMV and/or CMV infected cells and a circuit adapted for withdrawal of blood from the infected host. This circuit is in fluid connection with the collector, thus allowing withdrawn blood to be flowed into or through the collector where CMV and/or CMV infected cells are bound by the compound. The circuit may optionally be constructed to provide for the return of the purified blood to the host.

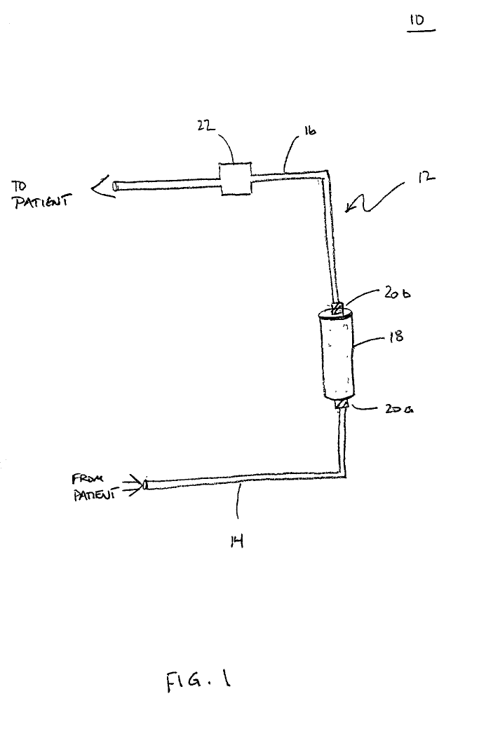

An exemplary apparatus that illustrates this general design is shown in FIG. 1. As can be seen, the collection apparatus 10 includes a fluid circuit 12 which itself includes an outlet line 14, a collector 18 and a return line 16. The circuit 12 is adapted to be connected to the blood system of the patient. Such connection can be made by any of a variety of techniques known in the art (e.g., by the use of needles or catheters attached to the outlet line 14 and return line 16). The outlet line 14 is adapted to allow for the withdrawal of blood from the patient; whereas, the return line 16 is adapted for blood flow back to the patient. The outlet line 14 and inlet line 16 are in fluid communication with the collector 18 and connected thereto via connectors 20a and 20b, respectively. Thus, the collector 18 containing the compound (not shown) is positioned within the circuit 12 such that blood withdrawn via outlet line 14 enters the collector 18 and purified blood leaves the collector for return to the patient via return line 16.

The circuit 12 can further include an optional pump 22 for facilitating the withdrawal and the return of the blood through the circuit. The pump can be of a variety of designs that are known in the art including, for example, pressure pumps and peristaltic pumps. Other additional components can also optionally be incorporated into the collection apparatus 10. For example, a detector (not shown) can be incorporated to monitor the concentration of virus returning to the patient by the return line 16. All or a portion of the circuit 12 (e.g., the collector 18) can also be enclosed in, or in contact with, a heating system (not shown) to monitor and maintain the temperature of the blood at an appropriate physiological temperature level. The circuit 12 can also include a pressure monitor (not shown) to monitor the blood pressure level within the circuit and optionally a pressure relief valve (not shown) to release pressure within the circuit if the pressure exceeds a threshold limit. Additionally, one or more inlets may be included in the circuit 12 for introduction of various agents (e.g., pharmaceutical agents and buffers) into the circuit.

As set forth supra, compounds contained in the collector often are ligands for US28. Additional details regarding suitable compounds for use in such devices are provided in section IV. In certain designs, the compounds are placed directly into the collector and the collector retains the compounds. Alternatively, the compounds are linked to a solid support (e.g., beads, microspheres, nanoparticles and colloidal particles). Such supports are selected to be compatible with the blood flowing through the collector and to have chemistries that permit the compounds to be attached thereto. Exemplary materials include, but are not limited to, Sepharose-based materials and a variety of polymers used in solid phase chemistry and affinity chromatography that are known to those of skill in the art. In some instances, the compounds are joined to the support by any of a number of linkers (e.g., straight or branched chain-carbon linkers, heterocyclic linkers and peptide linkers). A number of such linkers are listed in, and available from, Pierce Chemical Company in Rockford, Ill.; other linkers are described in U.S. Pat. Nos. 4,671,958; 4,659,839; 4,414,148; and 4,669,784, for example. If linkers and supports are utilized, they are selected so as not to interfere with the binding of the compound to CMV or to a CMV infected cell.

2. Mode of Operation

In the case of a device that utilizes an external collector, blood is withdrawn from the CMV infected host and flowed into or through the collector that contains the compound. The withdrawn blood once purified can then optionally be recirculated back to the host. While the apparatus shown in FIG. 1 illustrates a recirculating design, non-circulating designs can also be utilized. For example an apparatus can also be utilized in which blood is simply withdrawn into the circuit and flowed into the collector for collection of CMV or CMV infected cells (i.e., there is no return line 16; in this instance, a pump would Is typically be included to be in communication with the outlet line 14). Optionally, flow can then be reversed, with the purified blood reentering the host via the same line in which the blood was withdrawn. If a recirculating design is utilized as shown in FIG. 1, blood flow through the circuit 12 can be continuous or non-continuous. In continuous mode, removal of -blood from the host via the outlet line 14 occurs simultaneously with the return of treated blood from the collector 18 into the patient via the return line 16. In certain non-continuous methods, blood is initially flowed into the collection device 18 where the blood is allowed to reside, thereby providing for binding between the compound and CMV and CMV infected cells. Following this residence time, blood can then be passed from the collector 18 back to the host, for example.

C. Variations

In addition to the foregoing apparatus and devices, another design utilizes a patch that is impregnated or saturated with compound that binds CMV or CMV infected cells (e.g., US28 ligands). The patch is designed for attachment to an exterior skin surface. Contact between the compound and CMV and/or CMV infected cells in this approach is indirect. For example, compound can diffuse through the skin where the compound can bind CMV or CMV infected cells, thus resulting in the concentration of virus or virus infected cells in the vicinity of the patch. Alternatively, virus may be drawn through the skin (e.g., through pores in the skin) to the patch.

It should be appreciated that the apparatus and methods disclosed herein are not limited to use with humans. As pointed out in the overview, CMV infects a diverse group of animals, including most mammals and some non-mammals. Thus, the methods disclosed herein are useful for collecting CMV or CMV infected cells from mammals, including but not limited to, humans, non-human primates (e.g., monkeys, apes, gorillas and baboons), and a variety of commercial livestock (e.g., chickens, porcines, sheep and bovines).

IV. Compounds

The compounds utilized in the apparatus and methods of the invention are It;, ones that are able to bind to CMV or CMV infected cells. In some instances, such compounds are selected to bind to expression products of viral dissemination genes, i.e., genes that encode for proteins that are involved in viral dissemination in the host following infection. As alluded to supra, the evidence indicates that US28 encodes a receptor protein that binds chemokines and, in so doing, gains motility through the infected host. Thus, one class of compounds that are useful with the apparatus and methods disclosed herein are those in which the compound is a ligand for US28. Compounds suitable for the provided methods and apparatus, however, are not limited to this class of compounds. In general, the compounds can be proteins, peptides and small organic molecules (i.e., molecules with molecular weight of less than about 2000, sometimes less than 100, and in other instances less than 500). The compounds are also selected to have relatively high affinity for CVM or a CMV infected cell. For example, certain compounds have an EC₅₀ for binding to US28 of about 100 nM or less.

Exemplary molecules that exhibit high affinity for US28 and thus are suitable for use with the present methods and apparatus are described below. Further details regarding such molecules are set forth in PCT Application No. 01/27363, filed on Aug. 30, 2001, entitled “Modulators of US28,” and having attorney docket number 019934-00310PC, and in PCT Application No.01/27269, also filed on Aug. 30, 2001, entitled “Reagents and Methods for the Diagnosis of CMV Dissemination,” and having attorney docket number 019934-000910PC.

In one group of embodiments, the compounds have the formula:

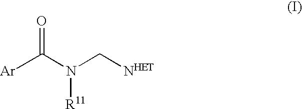

or a pharmaceutically acceptable salt thereof; wherein Ar represents a substituted aryl group; R¹¹ represents H or (C₁-C₄)alkyl; and N^{Het} is a substituted or unsubstituted 4-, 5-, 6-, or 7-membered nitrogen heterocycle.

Exemplary molecules within this group are those compounds having the formula:

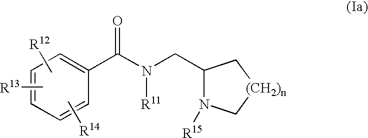

or a pharmaceutically acceptable salt thereof; wherein the subscript n is an integer of from 1 to 3; R¹¹ and R¹⁵ are independently selected from H and substituted or unsubstituted (C₁-C₄)alkyl; R¹², R¹³ and R¹⁴ are each members independently selected from H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; with the proviso that at least one of R¹², R¹³ and R¹⁴ is other than H.

For some compounds within this group, n is 1, R¹¹ is H, R¹⁵ is (C₁-C₄)alkyl; and R¹², R¹³ and R¹⁴ are each other than H. In other preferred embodiments, n is one; R¹¹ is H; R¹², R¹³ and R¹⁴ are each independently selected from H, hydroxy, halogen, (C₁-C₄)alkyl and (C₁-C₄)alkoxy; and R¹⁵ is (C₁-C₄)alkyl.

The current inventors have also discovered that S(−)-3-Iodo-2-hydroxy-6-methoxy-N[(1-ethyl-2-pyrrolidinyl)methyl]benzamide (S(−)-IBZM or IBZM, from the RBI 20 division of Sigma-Aldrich) is an effective inhibitor of the binding of native chemokine ligands (such as fractalkine and eotaxin, among others), to US28. Moreover, this compound was found to bind specifically to US28 among all chemokine receptors tested. Thus, this compound is useful in the methods and apparatus provided herein.

In another group of compounds, the compounds have the formula:

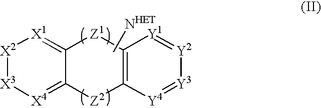

wherein X¹, X², X³ and X⁴ are each independently N or C—R¹, wherein R¹ is H, halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, or di(C₁-C₄)alkylamino. Similarly, Y¹, y², Y³ and Y⁴ are each independently N or C—R², wherein R² is H, halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, or di(C₁-C₄)alkylamino.

The symbol Z¹ represents a substituted or unsubstituted (C₁-C₃)alkylene. The symbol Z² represents a divalent moiety selected from —O—, —S—and —N(R)—wherein R is H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, or di(C₁-C₄)alkylamino.

The symbol N^{Het} represents a substituted or unsubstituted 4-, 5-, 6-, or 7-membered nitrogen heterocycle.

For certain compounds within this particular group, at least two of X¹, X², X³ and X⁴ are CH, more preferably three of X¹, X², X³ and X⁴ are CH and the fourth is C—R¹, wherein R¹ is halogen, (C₁-C₄)alkylthio, (C₁-C₄)haloalkyl, (C₁-C₄)haloalkoxy, nitro, cyano, or (C₁-C₄)acyl. Also preferred are those embodiments in which Y¹, Y², Y³ and Y⁴ are each independently C—R², wherein R² is H, halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, di(C₁-C₄)alkylamino. More preferably, each of Y¹, Y², Y³ and Y⁴ are independently C—R², wherein R² is H, halogen, (C₁-C₄)alkylthio, or (C₁-C₄)haloalkyl.

In other embodiments, Z¹ represents an ethylene or propylene group, more preferably an ethylene group in which N^{Het} is attached at the position adjacent to the ring defined by Y¹, Y², Y³ and Y⁴.

In still other compounds of this group, Z² is —O—or —S—, more preferably —S—.

Groups for N^{Het} are the substituted or unsubstituted 5—or 6-membered nitrogen heterocycles. In certain compounds, heterocycles include piperidine, piperazine, pyrrolidine, oxazoline, imidazoline, pyrazine and morpholine. More preferably, N^{Het} is a substituted or unsubstituted 6-membered nitrogen heterocycle. In other compounds, N^{Het} is a substituted or unsubstituted piperazine that is attached to Z¹ through a nitrogen atom of the piperazine ring. Useful substituents for the piperazine ring are (C₁-C₄)alkyl, (C₁-C₄)haloalkyl, or (C₁-C₄)acyl. Additional substitutents are (C₁-C₄)alkyl, with methyl, ethyl and propyl substituents preferred.

In still other instances, the compounds are substituted 10-piperazino-10,11-dihydrodibenzo(b,f)thiepins having the formula:

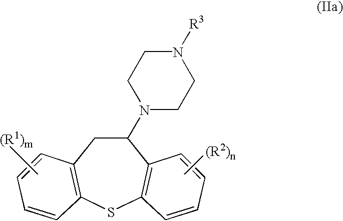

wherein the subscripts m and n are independently integers of from 0 to 3, preferably 0 to 2, more preferably 0 or 1; and R¹ and R² are substituents independently selected from the group of halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, and di(C₁-C₄)alkylamino. The symbol R³ represents (C₁-C₄)alkyl, (C₁-C₄)haloalkyl, or (C₁-C₄)acyl.

In certain of the compounds from this group, m is 0 and n is 1. For example, in some instances, m is 0, n is 1 and R² is selected from the group of halogen, (C₁-C₄)alkyl, (C₁-C₄)alkoxy, (C₁-C₄)alkylthio and (C₁-C₄)haloalkyl. In still other instances, m is 0, n is 1 and R² is selected from the group of halogen and (C₁-C₄)alkylthio. Most preferably, the R² substituent is at the 8-position of the dihydrodibenzo(b,f)thiepin ring system.

Additional compounds for use with the apparatus and methods disclosed herein are methiothepin (free base or salt, CAS No. 20229-30-5) and octoclothepin (free base or salt, CAS No. 4789-68-8, for the maleate salt).

Other suitable compounds for use with the apparatus and methods that are provided are described in U.S. Pat. No. 3,379,729 “Piperazinyldibenzothiepins” Apr. 23, 1968. See also U.S. Pat. No. 4,444,778. Still other related and useful dihydrodibenzo(b,f)thiepins are described in Jilek, et al., Collect. Czech. Chem. Commun. 33(6):1831-1845 (1968).

V. Exemplary Utilities

As alluded to in the foregoing sections, the apparatus and methods provided herein have utility in collecting or concentrating CMV and/or CMV infected cells in vivo or in vitro. As such, the methods and apparatus can be utilized in various treatment protocols to reduce the presence of CMV and CMV infected cells within an infected host. Such methods are expected to be useful, for example, in reducing blood viral load (with concomitant attenuation in pathology) in immuno-suppressed patients. Specific examples of such patients include those that are HIV positive and transplant recipients (e.g., individuals having undergone renal or soft organ transplants).

The methods can also be utilized to measure mutation rates and selective pressures after in vivo passage. In general, such methods involve collecting CMV utilizing the methods and apparatus disclosed herein. A segment of the viral genome is then analyzed to detect the presence or absence of a mutation (e.g., a point mutation, a deletion, an insertion, or a duplication). Assessment of mutation can be performed utilizing protocols that are known to those of skill in the art. For example, one can conduct genome restriction fragment length polymorphism analysis (RFLP), polymerase chain reaction (PCR) and RFLP across a segment potentially containing a mutation, and then culture and conduct in vitro drug resistance analysis of viral isolates.

Such analyses can be used to assess the emergence of, for instance, drug resistant populations of patients that are being treated with one or more pharmaceutical agents (e.g., cidofovir), before clinical signs of resistance are manifested. Additionally, the methodology provided herein can be utilized to detect reversion of recombinant virus (e.g., recombinant virus utilized as a vaccine; see, e.g., PCT Patent Application No. 01/27392, filed Aug. 31, 2001, entitled “Inhibition of CMV Infection and Dissemination,” and having attorney docket number 019934-002510PC). This provides the opportunity for a more timely therapeutic intervention. Thus, certain methods involve detecting the presence or absence of a mutation in mutational hotspots for the particular pharmaceutical agent of interest. If a mutation is detected, viral isolates can be cultured and examined for resistance. In certain analyses, patients receive the pharmaceutical agent before CMV is collected.

As with the other methods disclosed herein, analyses of this type are not limited to humans. Instead, such analyses can be conducted with other animals that are susceptible to CMV infection. Such animal studies can serve as useful models of human CMV infection and treatment.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes to the same extent as if each individual publication, patent or patent application were specifically and individually indicated to be so incorporated by reference.

What is claimed is:

1. An apparatus for collection of cytomegalovirus (CMV) or a CMV infected cell, the apparatus comprising:
(a) a collector comprising a compound that binds CMV or the CMV infected cell; and
(b) a circuit (i) adapted for connection to the blood system of a patient, (ii) adapted for the flow of withdrawn blood therethrough, and (iii) in fluid communication with the collector.

2. The apparatus of claim 1, wherein
the circuit comprises an outlet line and a return line, each in fluid communication with the blood system of the patient; and
the circuit is adapted for withdrawal of blood from the patient's blood system via the outlet line, passage of the blood through the collector and the return of the blood to the blood system via the return line.

3. The apparatus of claim 1, wherein the compound is contained on a support within the collector.

4. The apparatus of claim 3, wherein the support is selected from the group consisting of beads, microspheres, nanoparticles and-colloidal particles.

5. The apparatus of claim 2, further comprising a pump adapted to pump blood through the circuit.

6. The apparatus of claim 1, wherein the compound is a ligand for CMV US28.

7. The apparatus of claim 6, wherein the compound has the formula: 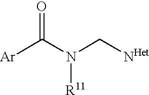
or is a pharmaceutically acceptable salt thereof; and wherein Ar is a substituted aryl group;
R¹¹ is a member selected from the group consisting of H and substituted or unsubstituted (C₁-C₄)alkyl; and
N^{Het} is a substituted or unsubstituted 4-, 5-, 6-, or 7-membered nitrogen heterocycle.

8. The apparatus of claim 6, wherein the compound has the formula: 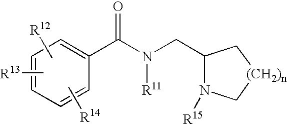
or is a pharmaceutically acceptable salt thereof; and wherein
the subscript n is an integer of from 1 to 3;
R¹¹ and R¹⁵ are members independently selected from the group consisting of H and substituted or unsubstituted (C₁-C₄)alkyl;
R¹², R¹³ and R¹⁴ are each members independently selected from the group consisting of H, hydroxy, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino and di(C₁-C₄)alkylamino;
with the proviso that at least one of R¹², R¹³ and R¹⁴ is other than

9. The apparatus of claim 6, wherein the compound has the formula: 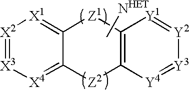
or is a pharmaceutically acceptable salt thereof; and wherein
X¹, X², X³ and X⁴ are each independently members selected from the group consisting of N and C—R¹, wherein R¹ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Y¹, Y², Y³ and Y⁴ are each independently members selected from the group consisting of N and C—R², wherein R² is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Z¹ is a divalent moiety selected from the group consisting of (C₁-C₃)alkylene;
Z² is a divalent moiety selected from the group consisting of —O—, —S—and —N(R³)— wherein R³ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
N^{Het} is a substituted or unsubstituted 4-, 5-, 6-, or 7-membered nitrogen heterocycle.

10. The apparatus of claim 6, wherein the compound has the formula: 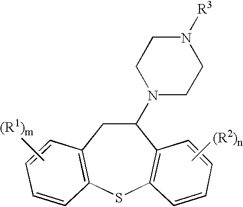
or is a pharmaceutically acceptable salt thereof; and wherein
the subscripts m and n are independently integers of from 0 to 3;
S R¹ and R² are substituents independently selected from the group consisting of halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamnino, and di(C₁-C₄)alkylamino; and
R³ is a substituent selected from the group consisting Of (C₁-C₄)alkyl, (C₁-C₁-C₄)haloalkyl and (C₁-C₄)acyl.

11. A device for collecting CMV or a CMV infected cell, the apparatus comprising a support and a compound that binds CMV and/or the CMV infected cell.

12. The device of claim 111, wherein the support is an implant device adapted for insertion into a patient.

13. The device of claim 12, wherein the implant device is adapted to be in contact with the blood of the patient.

14. The device of claim 1, wherein the implant comprises an absorptive material.

15. The device of claim 14, wherein the absorptive material is a surgical sponge.

16. The device of claim 15, wherein the absorptive material is made of a material selected from the group consisting of gelfoam, polyester and polyurethane.

17. The device of claim 11, wherein the support is a patch adapted for application to skin.

18. The device of claim 11, wherein the compound is a ligand for CMV US28.

19. The device of claim 18, wherein the compound has the formula: 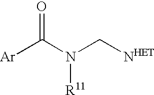
or is a pharmaceutically acceptable salt thereof; wherein
Ar is a substituted aryl group;
R¹¹ is a member selected from the group consisting of H and substituted or unsubstituted (C₁-C₄)alkyl; and
N^{Het} is a substituted or unsubstituted 4-, 5-, 6-, or 7-membered nitrogen heterocycle.

20. The device of claim 18, wherein the compound has the formula: 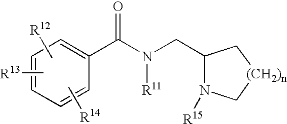
or is a pharmaceutically acceptable salt thereof; and wherein
the subscript n is an integer of from 1 to 3;
R¹¹ and R¹⁵ are members independently selected from the group consisting of H and substituted or unsubstituted (C₁-C₄)alkyl;
R¹², R¹³ and R¹⁴ are each members independently selected from the group consisting of H, hydroxy, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino and di(C₁-C₄)alkylamino;
with the proviso that at least one of R¹², R¹³ and R¹⁴ is other than H.

21. The apparatus of claim 18, wherein the compound has the formula: 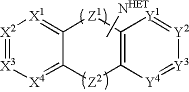
or is a pharmaceutically acceptable salt thereof; and wherein
X¹, X², X³ and X⁴ are each independently members selected from the group consisting of N and C—R¹, wherein R¹ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Y¹, Y², Y³ and Y⁴ are each independently members selected from the group consisting of N and C—R², wherein R² is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Z¹ is a divalent moiety selected from the group consisting of (C₁-C₃)alkylene;
Z² is a divalent moiety selected from the group consisting of —O—, —S—and —N(R³)—wherein R³ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
NHet is a substituted or unsubstituted 4-, 5-, 6-, or 7-membered nitrogen heterocycle.

22. The device of claim 18, wherein the compound has the formula: 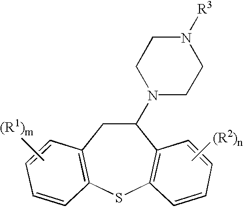
or is a pharmaceutically acceptable salt thereof; and wherein
the subscripts m and n are independently integers of from 0 to 3;
R¹ and R² are substituents independently selected from the group consisting of halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
R³ is a substituent selected from the group consisting of (C₁-C₄)alkyl, (C₁-C₄)haloalkyl and (C₁-C₄)acyl.

23. A method for collecting cytomegalovirus (CMV) or a CMV infected cell from a patient infected with CMV, the method comprising inserting a support comprising a compound that binds CMV and/or the CMV infected cell into the patient's blood system, whereby CMV or a CMV infected cell in the blood is collected at the support.

24. The method of claim 23, further comprising removing the implant device from the patient after CMV and/or CMV infected cells have accumulated at the implant device.

25. The method of claim 22, wherein the compound is a ligand for CMV US28.

26. The method of claim 25, wherein the compound has the formula: 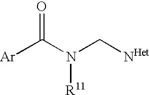
or is a pharmaceutically acceptable salt thereof; wherein
Ar is a substituted aryl group;
R¹¹ is a member selected from the group consisting of H and substituted or unsubstituted (C₁-C₄)alkyl; and
N^{Het} is a substituted or unsubstituted 4-, 5-, 6-, or 7-membered nitrogen heterocycle.

27. The method of claim 25, wherein the compound has the formula: 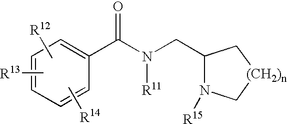
or is a pharmaceutically acceptable salt thereof; and wherein
the subscript n is an integer of from 1 to 3;
R¹¹ and R¹⁵ are members independently selected from the group consisting of H and substituted or unsubstituted (C₁-C₄)alkyl;
R¹², R¹³ and R¹⁴ are each members independently selected from the group consisting of H, hydroxy, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino and di(C₁-C₄)alkylamino;
with the proviso that at least one of R¹², R¹³ and R¹⁴ is other than H.

28. The method of claim 25, wherein the compound has the formula: 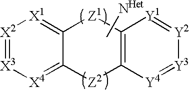
or is a pharmaceutically acceptable salt thereof; and wherein
X¹, X², X³ and X⁴ are each independently members selected from the group consisting of N and C—R¹, wherein R¹ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Y¹, Y², Y³ and Y⁴ are each independently members selected from the group consisting of N and C—R², wherein R² is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Z¹ is a divalent moiety selected from the group consisting of (C₁-C₃)alkylene;
Z² is a divalent moiety selected from the group consisting of —O—, —S—and —N(R³)— wherein R³ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
N^{Het} is a substituted or unsubstituted 4-, 5-, 6-, or 7-membered nitrogen heterocycle.

29. The method of claim 25, wherein the compound has the formula: 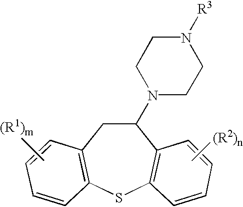
or is a pharmaceutically acceptable salt thereof; and wherein
the subscripts m and n are independently integers of from 0 to 3;
R¹ and R² are substituents independently selected from the group consisting of halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
R³ is a substituent selected from the group consisting of (C₁-C₄)alkyl, (C₁-C₄)haloalkyl and (C₁-C₄)acyl.

30. The method of claim 22, wherein the patient is a mammal.

31. The method of claim 30, wherein the patient is a non-human mammal.

32. A method for collecting cytomegalovirus (CMV) or a CMV infected ail cell from a patient infected with CMV, the method comprising contacting the patient's blood or a tissue that contains CMV or a CMV infected cell with a compound that binds CMV, whereby CMV or the CMV infected cell in the blood or the tissue is collected by the compound.

33. The method of claim 32, wherein contacting comprises contacting the patient's blood with the compound.

34. The method of claim 33, wherein contacting comprises withdrawing blood containing CMV or the CMV infected cell from the patient and flowing the blood through or into a collector that contains the compound, whereby CMV and/or the CMV infected cell binds to the compound in the collector.

35. The method of claim 34, further comprising recirculating the blood back into the patient after the contacting step.

36. The method of claim 35, wherein the withdrawing, contacting and recirculating steps are performed continuously.

37. The method of claim 32, wherein the collector comprises a support material that contains the compound.

38. The method of claim 32, wherein the compound is a ligand for CMV US28.

39. The method of claim 38, wherein the compound has the formula: 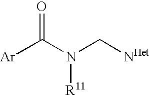
or is a pharmaceutically acceptable salt thereof; wherein
Ar is a substituted aryl group;
R¹¹ is a member selected from the group consisting of H and substituted or unsubstituted (C₁-C₄)alkyl; and
N^{Het} is a substituted or unsubstituted 4-, 5-, 6-, or 7-membered nitrogen heterocycle.

40. The method of claim 38, wherein the compound has the formula: 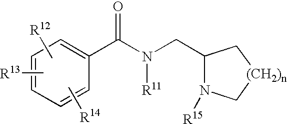
or is a pharmaceutically acceptable salt thereof; and wherein
the subscript n is an integer of from 1 to 3;
R¹¹ and R¹⁵ are members independently selected from the group consisting of H and substituted or unsubstituted (C₁-C₄)alkyl;
R¹², R¹³ and R¹⁴ are each members independently selected from the group consisting of H, hydroxy, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino and di(C₁-C₄)alkylamino;
with the proviso that at least one of R¹², R¹³ and R¹⁴ is other than H.

41. The method of claim 38, wherein the compound has the formula: 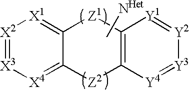
or is a pharmaceutically acceptable salt thereof; and wherein
X¹, X², X³ and X⁴ are each independently members selected from the group consisting of N and C—R¹, wherein R¹ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Y¹, Y¹, Y³ and Y⁴ are each independently members selected from the group consisting of N and C—R², wherein R² is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Z¹ is a divalent moiety selected from the group consisting of (C₁-C₃)alkylene;
Z² is a divalent moiety selected from the group consisting of —O—, —S—and —N(R³)—wherein R³ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and N^{Het} is a substituted or unsubstituted 4-, 5-, 6-, or 7-membered nitrogen heterocycle.

42. The method of claim 38, wherein the compound has the formula: 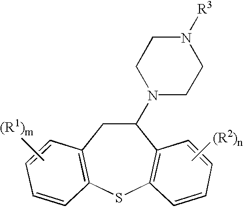
or is a pharmaceutically acceptable salt thereof; and wherein
the subscripts m and n are independently integers of from 0 to 3;
R¹ and R² are substituents independently selected from the group consisting of halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
R³ is a substituent selected from the group consisting of (C₁-C₄)alkyl, (C₁-C₄)haloalkyl and (C₁-C₄)acyl.

43. The method of claim 32, wherein contacting comprises placing a patch containing the compound on the skin of the patient.

44. The method of claim 32, further comprising providing a collection apparatus, the collection apparatus comprising the collector and a circuit, wherein
the circuit (i) comprises an outlet line and a return line, (ii) is adapted for connection to the blood system of a patient and the flow of withdrawn blood therethrough, and (iii) is in fluid communication with the collector;
withdrawing comprises withdrawing blood from the patient via the outlet line and flowing withdrawn blood through the collector; and
recirculating comprises recirculating the blood back to the patient via the return line.

45. The method of claim 32, wherein the patient is a mammal.

46. The method of claim 44, wherein the patient is a non-human mammal.

47. A method for assessing mutations in cytomegalovirus (CMV), the method comprising:
(a) collecting CMV and/or at least one CMV infected cell from a patient infected with CMV by contacting the blood or a tissue of the patient with a compound that binds CMV and/or a CMV infected cell, whereby CMV or at least one CMV infected cell is bound from the blood or tissue; and
(b) detecting the presence and/or absence of a mutation in CMV obtained from the CMV or the at least one CMV infected cell collected in step (a).

48. The method of claim 47, wherein contacting comprises withdrawing blood containing CMV from the patient and flowing the blood into or through a collector that comprises the compound, whereby CMV in the blood is captured by the compound of the collector.

49. The method of claim 48, wherein the compound is a ligand for CMV US28.

50. The method of claim 49, wherein the compound has the formula: 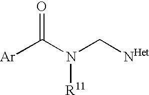
or is a pharmaceutically acceptable salt thereof; wherein
Ar is a substituted aryl group;
R¹¹ is a member selected from the group consisting of H and substituted or unsubstituted (C₁-C₄)alkyl; and
N^{Het} is a substituted or unsubstituted 4-, 5-, 6-, or 7-membered nitrogen heterocycle.

51. The method of claim 49, wherein the compound has the formula: 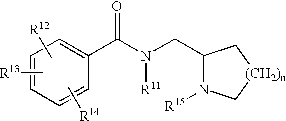
or is a pharmaceutically acceptable salt thereof; and wherein
the subscript n is an integer of from 1 to 3;
R¹¹ and R¹⁵ are members independently selected from the group consisting of H and substituted or unsubstituted (C₁-C₄)alkyl;
R¹², R¹³ and R¹⁴ are each members independently selected from the group consisting of H, hydroxy, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino and di(C₁-C₄)alkylamino;
with the proviso that at least one of R¹², R¹³ and R¹⁴ is other than H.

52. The method of claim 49, wherein the compound has the formula: 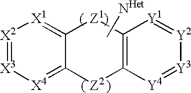
or is a pharmaceutically acceptable salt thereof; and wherein
X¹, X², X³ and X⁴ are each independently members selected from the group consisting of N and C—R¹, wherein R¹ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Y¹, Y², Y³ and y⁴ are each independently members selected from the group consisting of N and C—R², wherein R² is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Z¹ is a divalent moiety selected from the group consisting of (C₁-C₃)alkylene;
Z² is a divalent moiety selected from the group consisting of —O—, —S—and —N(R³)—wherein R³ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
N^{Het} is a substituted or unsubstituted 4-, 5-, 6-, or 7-membered nitrogen heterocycle.

53. The method of claim 49, wherein the compound has the formula: 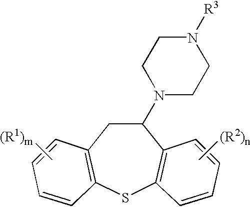
or is a pharmaceutically acceptable salt thereof; and wherein
the subscripts m and n are independently integers of from 0 to 3;
R¹ and R² are substituents independently selected from the group consisting of halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylainino, and
di(C₁-C₄)alkylamino; and
R³ is a substituent selected from the group consisting of (C₁-C₄)alkyl, (C₁-C₄)haloalkyl and (C₁-C₄)acyl.

54. The method of claim 47, wherein collecting comprises placing an implant device that contains the compound in or on the patient such that the implant device is in contact with the blood of the patient, whereby CMV or the at least one CMV infected cell in the blood is captured by the compound of the implant device.

55. The method of claim 54, wherein the compound is a ligand for CMV US28.

56. The method of claim 55, wherein the compound has the formula: 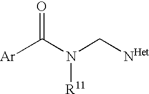
or is a pharmaceutically acceptable salt thereof; wherein
Ar is a substituted aryl group;
R¹¹ is a member selected from the group consisting of H and substituted or unsubstituted (C₁-C₄)alkyl; and
N^{Het} is a substituted or unsubstituted 4-, 5-, 6-, or 7-membered nitrogen heterocycle.

57. The method of claim 55, wherein the compound has the formula: 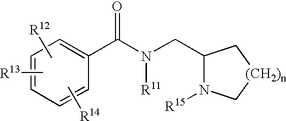
or is a pharmaceutically acceptable salt thereof; and wherein the subscript n is an integer of from 1 to 3;
R¹¹ and R¹⁵ are members independently selected from the group consisting of H and substituted or unsubstituted (C₁-C₄)alkyl;
R¹², R¹³ and R¹⁴ are each members independently selected from the group consisting of H, hydroxy, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino and di(C₁-C₄)alkylamino;
with the proviso that at least one of R¹², R¹³ and R¹⁴ is other than H.

58. The method of claim 55, wherein the compound has the formula: 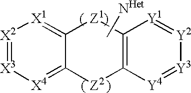
or is a pharmaceutically acceptable salt thereof; and wherein
X¹, X², X³ and X⁴ are each independently members selected from the group consisting of N and C—R¹, wherein R¹ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Y¹, Y², Y³ and Y⁴ are each independently members selected from the group consisting of N and C—R², wherein R² is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino;
Z¹ is a divalent moiety selected from the group consisting of (C₁-C₃)alkylene;
Z² is a divalent moiety selected from the group consisting of —O—, —S—and —N(R³)— wherein R³ is a member selected from the group consisting of H, halogen, (C₁-C₄)alkyl, (C₁-C₄)alkoxy, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
N^{Het} is a substituted or unsubstituted 4-, 5-, 6-, or 7-membered nitrogen heterocycle.

59. The method of claim 55, wherein the compound has the formula: 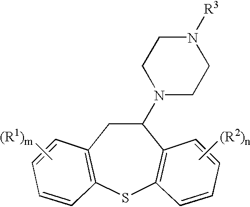
or is a pharmaceutically acceptable salt thereof; and wherein
the subscripts m and n are independently integers of from 0 to 3;
R¹ and R² are substituents independently selected from the group consisting of halogen, (C₁-C₄)alkyl, (C₁-C₄)alkoxy, (C₁-C₄)alkylthio, (C₁-C₄)haloalkyl, (C₁-C₄)haloalkoxy, nitro, cyano, (C₁-C₄)acyl, amino, (C₁-C₄)alkylamino, and di(C₁-C₄)alkylamino; and
R³ is a substituent selected from the group consisting of (C₁-C₄)alkyl, (C₁-C₄)haloalkyl and (C₁-C₄)acyl.

60. The method of claim 47, wherein if a mutation is detected, the method further comprises determining whether the mutation confers resistance to a pharmaceutical agent.

61. The method of claim 60, wherein the method further comprises administering the pharmaceutical compound to the patient prior to the collection step.

62. The method of claim 47, wherein collecting comprises placing a transdermal patch containing the compound on the skin of the patient.
